(19) United States
(12) Patent Application Publication
(10) Pub. No.: US 2016/0175083 A1
(43) Pub. Date: Jun. 23, 2016

(54) MIXED USE FIXATION ELEMENTS FOR ENDOLUMINAL FILTERS

(71) Applicant: VOLCANO CORPORATION, San Diego, CA (US)

(72) Inventors: Eric Johnson, Woodside, CA (US); Jeremy Stigall, Carlsbad, CA (US)

(21) Appl. No.: 14/958,234

(22) Filed: Dec. 3, 2015

Related U.S. Application Data
(60) Provisional application No. 62/093,969, filed on Dec. 18, 2014.

(51) Int. Cl. A61F 2/01

(52) U.S. Cl. CPC A61F 2/01; A61F 2002/016; A61F 2220/0016; A61F 2210/0004; A61F 2002/011

(57) ABSTRACT

Filters and methods of deploying and retrieving filters are described. The filters can include a first support member and second support member that form a crossover. A material capture structure can also extend between the first and second support members. Permanent and degradable fixation elements or tissue anchors on either or both of the support members can also be included. The fixation elements can also be retractable.

### CROSS REFERENCE TO RELATED APPLICATIONS

This application claims priority to and the benefit of U.S. Provisional Application No. 62/093,969, filed on Dec. 18, 2014, the content of which is incorporated herein in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This application relates to the field of endoluminal filters having more than one type of fixation elements to secure the filter in the desired position. The types of fixation elements may include permanent fixation elements and temporary fixation elements. Temporary fixation elements may include one or more biodegradable, bioabsorbable or bioresorbable fixation elements or retractable fixation elements. The different types of fixation elements may be used in various combinations depending upon a variety of factors.

### BACKGROUND

FIGS. 1A, 1B and 1C illustrate a recovery sequence of filter positioned within a lumen and having a plurality of fixation elements within the luminal walls. In FIG. 1A, the filter is fully deployed into the lumen with fixation elements on both loops engaged with the luminal walls. While removed for purposes of clarity in this figure, there would likely be a degree of incorporation of the filter support frame into the lumen walls. FIG. 1A also illustrates an initial step of retrieving the filter. The outer sheath 710, inner sheath 710i are positioned adjacent the retrieval feature with the snare over the retrieval feature 240. The snare 712 is shown in initial engagement with the retrieval feature 240. While shown engaging from within the inner sheath 710i, the snare 712 (along with the inner sheath) may extend from beyond the distal most end of the outer sheath 710 prior to engagement with the filter retrieval filter.

Next, as shown in FIG. 1B, the snare 712 is withdrawn into the inner sheath 710i collapsing the retrieval feature 240 in the process. With the filter secured against the inner sheath 710i, the outer sheath 710 is advanced over the filter as shown in FIGS. 1B and 1C. As the outer sheath 710 advances along the filter, the filter loops collapse and the support frames are pulled away from the lumen walls. The force needed to pull the support frame away from the wall must overcome both the tissue incorporating the support frame to the wall as well as the anchors penetrated into the wall.

While the anchors in the illustrated design are spaced along the support frame such that the anchors are removed sequentially, there is still considerable retrieval force required since the retrieval method must overcome both anchor forces and tissue incorporation forces. What is needed are improved intraluminal filter fixation systems to reduce the retrieval force needed during filter recovery.

### SUMMARY OF THE DISCLOSURE

The present invention relates to endoluminal filters having permanent and degradable fixation elements and methods of reducing forces needed when recovering filters after tissue incorporation has occurred between the filter and a luminal wall.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A, 1B and 1C illustrate the initial stages of a deployed intraluminal filter during initial capture (FIG. 1A), and stages of retrieval (FIGS. 1B and 1C).

FIG. 2 illustrates a perspective bottom up view of a filter having fixation elements of different configurations.

FIGS. 3A and 3B are perspective and cross section views respectively of a fixation element having a standard wire portion and a biodegradable, bioabsorbable or bioresorbable portion.

FIG. 4A illustrates a fixation element with a biodegradable, bioabsorbable or bioresorbable portion engaged with the side wall of lumen.

FIG. 4B illustrates an enlarged view of the tissue engagement element of FIG. 4A with a biodegradable, bioabsorbable or bioresorbable tip portion extending beyond the crimp.

FIG. 5A is a perspective view of an extended anchor shown in the first loop of the filter of FIG. 2 having a biodegradable, bioabsorbable or bioresorbable section A and B.

FIG. 5B is a side view of an alternative tip for the extended anchor of FIG. 5A.

FIGS. 6A, 6B and 6C illustrate a section view of a vessel with the filter of FIG. 2 disposed therein during capture (FIG. 6A), initial retrieval (FIG. 6B) and reduced effort second stage of recovery (FIG. 6C).

FIGS. 7A and 7B illustrate two stages of the retrieval on either end of a filter having biodegradable, bioabsorbable or bioresorbable anchors depending upon degree of incorporation.

FIG. 8 illustrates a double ended biodegradable, bioabsorbable or bioresorbable fixation element with different tip orientations attached to an elongate body.

FIG. 9A illustrates a tissue anchor having a biodegradable, bioabsorbable or bioresorbable base attached to a tube that is attached to a support member.

FIG. 9B illustrates a tissue anchor on a biodegradable, bioabsorbable or bioresorbable tube on a support member.

FIG. 9C illustrates a biodegradable, bioabsorbable or bioresorbable tissue anchor on a degradable base on a tube attached to a support member.

FIG. 9D illustrates a plurality of the tissue anchors illustrated in any of the FIGS. 9A, 9B or 9C positioned along a pair of support structures.

FIG. 10 is a variation of FIGS. 9A, 9B and 9C illustrating the use of a hook shaped fixation element.

FIG. 11 is a variation of FIG. 9B illustrating the alternative use of a biodegradable, bioabsorbable or bioresorbable wrap to replace the degradable tube.

FIGS. 12A and 12B illustrate section and perspective views respectively of a dual lumen sleeve enclosing a support and member and an anchor support wire.

FIG. 13 is a dual limen sleeve as in FIGS. 12A and 12B having multiple ports.

FIG. 14 is a perspective view of an anchor wire with three anchors.

FIGS. 15A and 15B are perspective and enlarged views of an endoluminal filter with three retractable anchors and 15B is the retraction and extension device on one end of the filter retracting or deploying the anchors.

FIG. 16 is a perspective view of a dual loop filter having multiple mixed retractable or biodegradable, bioabsorbable or bioresorbable fixation elements.

FIGS. 17A, 17B and 17C illustrate various filters having one or more biodegradable, bioabsorbable or bioresorbable anchors as described in FIGS. 3A-4B.

FIGS. 18A, 18B, and 18C illustrate various filters having one or more biodegradable, bioabsorbable or bioresorbable anchors integrally formed with the filter as described in FIGS. 5A and 5B.

FIG. 19A is a side view of a non-penetrating anchoring member positioned against a lumen wall.

FIG. 19B is an enlarged view of the adhesive layer engagement between the anchoring member and wall portion.

FIG. 19C is a top down view of the adhesive layer on the distal most end of the anchoring member of FIGS. 19A and 19B.

FIGS. 19D, 19E and 19F illustrate top down views of alternative configurations of the distal most end of the anchoring member illustrated in FIG. 19C.

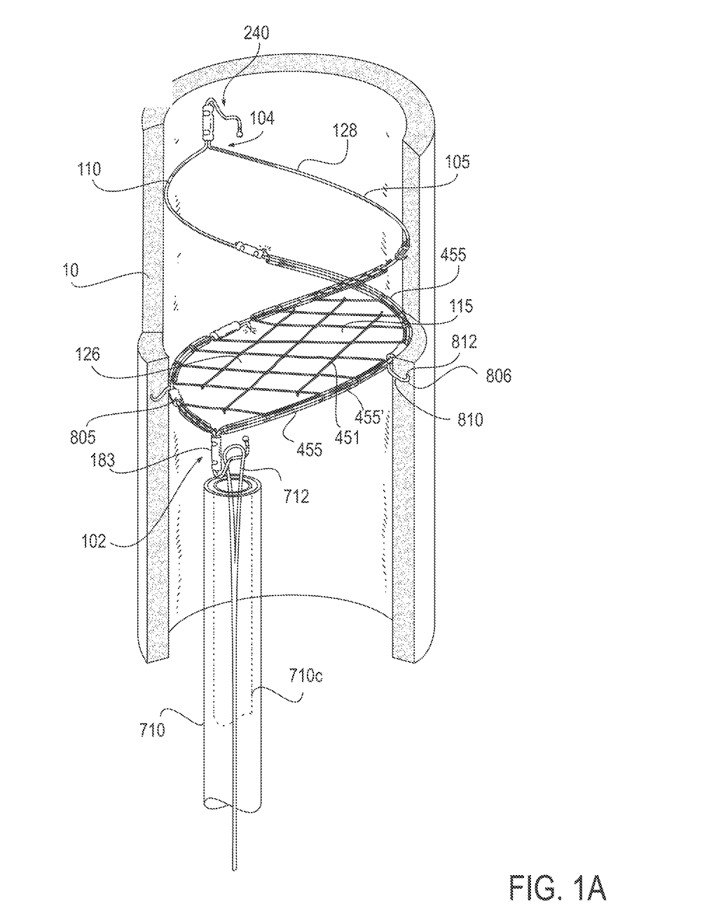

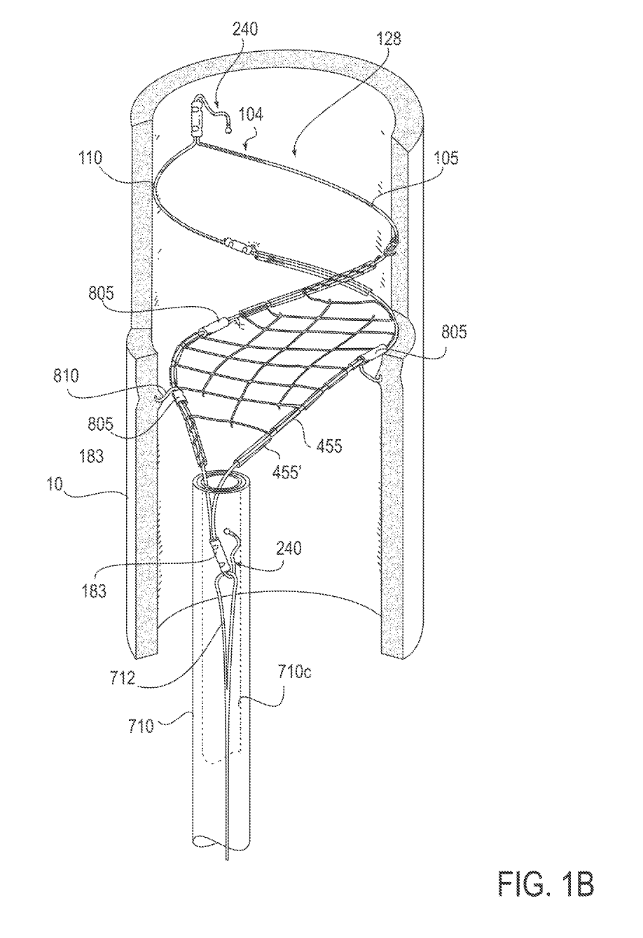

### DETAILED DESCRIPTION

Various details of the designs, materials and features of the filters described herein are available in U.S. patent application Ser. No. 11/325,230, filed Jan. 3, 2006, titled “ENDOLUMINAL FILTER,” now U.S. Pat. No. 7,854,747; U.S. patent application Ser. No. 13/553,335, filed Jul. 9, 2012, titled “ENDOLUMINAL FILTER WITH FIXATION,” Publication No. US-2013-0012981-A1; and International Patent Application No. PCT/US2013/021285, filed Jan. 11, 2013, Publication No. WO 2013/106746 A2, each of which is herein incorporated by reference its entirety.

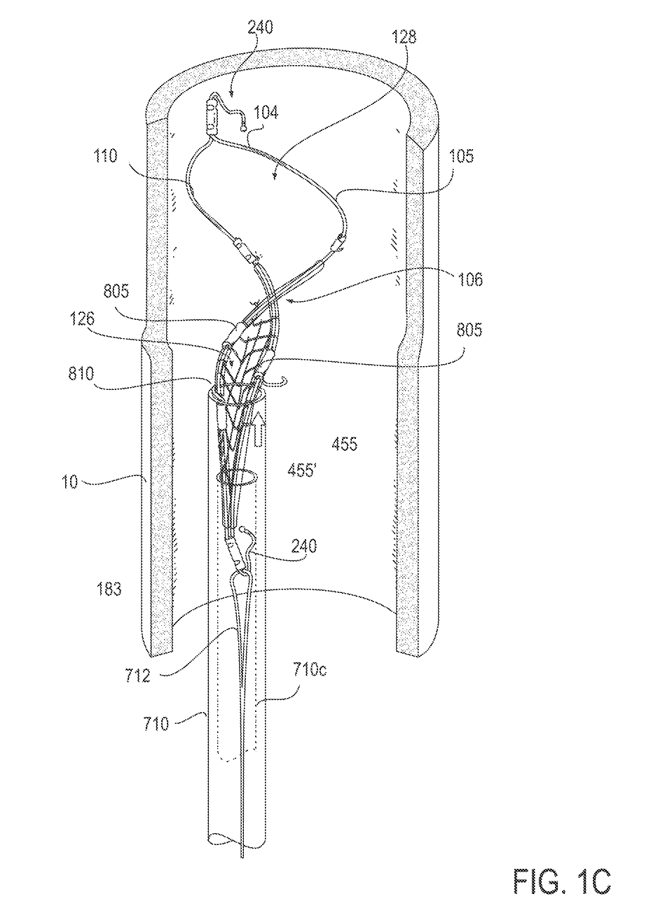

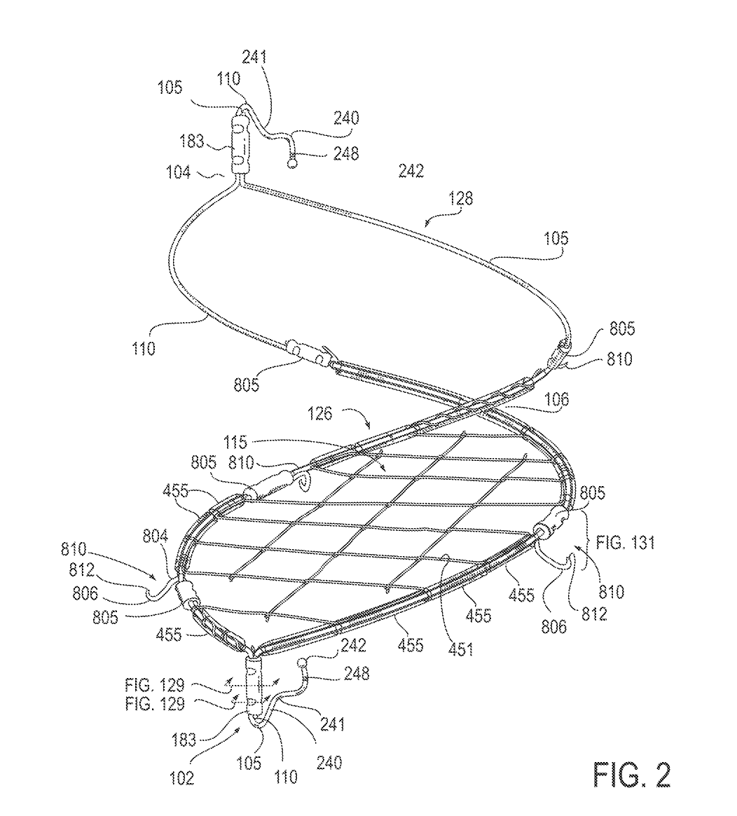

FIG. 2 illustrates a perspective bottom up view of an embodiment of a filter having fixation element of two different configurations. FIG. 2 is of similar construction to the filters described in the above incorporated applications. The FIG. 2 embodiment includes the use of two different configurations of fixation elements according to one aspect of the present invention. The types of fixation elements may include permanent fixation elements and temporary fixation elements. In various aspects of the invention, temporary fixation elements may include one or more biodegradable, bioabsorbable or bioresorbable fixation elements or retractable fixation elements. As will be appreciated in the description that follows, the different types of fixation elements may be used in various combinations depending upon a variety of factors. In one aspect, a permanent fixation element or anchor member is one that is deployed initially with the filter and is present when the filter is retrieved. A degradable fixation element may be entirely degradable or may have a permanent portion and a degradable portion. In one aspect, there is a degradable fixation element having a permanent portion that is used to attach the fixation element to a support member and a degradable portion beyond. After use, only the permanent portion attached to the support frame remains. Fixation elements including degradable portions can provide the ability to reduce a holding force between the filter and the implantation location (e.g., vessel wall). A reduced holding force can allow for easier removal of the filter as less force may be required to separate the filter from the implantation site.

Materials suited for use as temporary fixation element having the desired biodegradable, bioabsorbable or bioresorbable qualities include, for example, biodegradable polymers may also be used to form components of embodiments of the filter devices disclosed herein. For example, polylactide (PLA), a biodegradable polymer, has been used in a number of medical device applications including, for example, tissue screws, tacks, and suture anchors, as well as systems for meniscus and cartilage repair. A range of synthetic biodegradable polymers are available, including, for example, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(DTH iminocarbonate), poly(bisphenol A iminocarbonate), poly(ortho ester), polycyanoacrylate, and polyphosphazene. Additionally, a number of biodegradable polymers derived from natural sources are available such as modified polysaccharides (cellulose, chitin, dextran) or modified proteins (fibrin, casein). The most widely compounds in commercial applications include PGA and PLA, followed by PLGA, poly(e-caprolactone), polydioxanone, trimethylene carbonate, and polyanhydride. In addition, alternatively or optionally, some metals may be have material properties suited for use in one or more embodiments of the medical devices having temporary fixation elements described herein. Exemplary metals include a metal comprising an austenitic alloy, a magnesium alloy, Fe alloy or combinations thereof such as, for example, Mg—Al/Y/Li/RE, Mg—Ca, Mg—ZN—Mn.

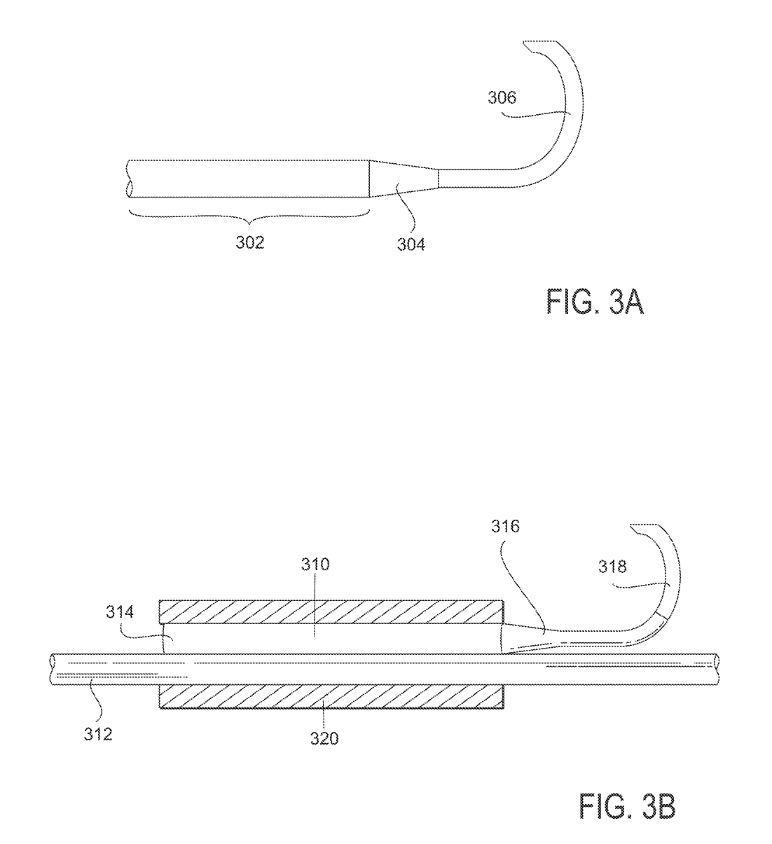

In the illustrative embodiment of FIG. 2, the second loop or frame 128 includes fixation elements similar to those shown and described below with regard to FIGS. 3A and 3B or, optionally as in other embodiments. The three fixation elements or anchors in the first loop or frame 126 are of the type described and illustrated in greater detail in FIGS. 130A-130C, 131, 132, 133 and 134 of PCT Publication No. US-2013-0012981.

The filter embodiment illustrated in FIG. 2 includes the elongate structural members 105, 110 joined using joints or connectors 183 at ends 102, 104. The support members 105, 110 have a spiral shape and cross at crossover 106. The ends of the support members are formed into retrieval features 240 having undulations 241, plasma ball tips 242 and marker bands 248 as described elsewhere in further detail.

In one embodiment, a medical device support structure includes two or more fixation elements, tissue anchors or tissue engagement structures of two different types—one is permanent and the other is temporary. As used herein a permanent fixation element is one that is deployed with the medical device and is recovered with the medical device from a position engaged with tissue during the implantation phase of medical device use. In contrast, a temporary fixation element is one that is present when the device is deployed but substantially disengages from either the tissue or the medical device when the medical device is recovered after use. In embodiments having retrievable fixation elements, those elements are substantially disengaged when withdrawn so as to be separated from the tissue even if not completely withdrawn into the medical device. In the case of bioabsorbable, biodegradable or bioresorbable fixation elements, substantial disengagement occurs when the fixation element has dissolved and been released from the medical device, been absorbed by the surrounding tissue and/or is structurally degraded to a hold force below that of any incorporated tissue that is resent on the medical device in the vicinity of the fixation element.

Numerous various alternative fixation elements, tissue anchors or tissue engagement structures are described above and may be adapted into a variety of combinations and configurations according to the embodiments that follow. FIG. 2 illustrates an endoluminal filter having a first support member 105 having a first end (adjacent 102 and formed into a retrieval feature 240) and a second end (adjacent 104 and straight ended in the attachment 183) and a second support member 110 with a second end (straight end at 105) attached to the first end of the first support member and a first end formed into a retrieval feature 240 at the device second end 104. In the illustrated embodiment, the first support member 105 and the second support member 110 are each formed from a single wire that extends from at least the first end 102 to the second end 104. The support members may extend beyond the end 102, 104 and be used to form retrieval features 240 or other elements of the filter as described below.

The filter of FIG. 2 has a retrieval feature 240 on the first end 102 and a retrieval feature 240 on the second end 104. The second support member 110 forms a crossover 106 with the first support member 105. In one embodiment, the second support member 110 is attached to the first end of the first support member 102 and the second end of the first support member 104. A material capture structure 115 extends between the first and second support members 105, 110, the crossover 106 the first end or the second end of the first support member 105. In the illustrated embodiment, the material capture structure is located within the first loop or frame 126 that is between the first and second support structures 105, 110, the first end 102 and the cross over 106. A second loop or frame 128 is formed between the first and second support structures 105, 110, the second end 104 and the cross over 106.

In the illustrated embodiment of FIG. 2, five tissue anchors are provided on support members 105, 110. Specifically, there are three extended tissue anchors of a first configuration in the first loop and two tissue anchors of a second different configuration in the second loop. There are two extended tissue anchors on the support member 105 and one extended tissue anchor on the support member 110. All five tissue anchors are arranged in an offset configuration so that when stowed prior to deployment or after retrieval, the anchors are aligned in proximal and distal orientation instead of being side to side. Offset alignment is further described above with regard to FIG. 91 of Publication No. US-2013-0012981-A1. The three extended tissue anchors in the first loop are offset so that the third anchor (attached to support member 110) will be positioned between the first and second anchors (attached to support member 105). Similarly, the anchors in the second loop are offset so that the anchor on the support member 105 will be closer to the cross over 106 when stowed or upon retrieval. As described herein, there is a combination of permanent and temporary anchor types used in this illustrative embodiment.

FIGS. 3A and 3B are perspective and cross section views respectively of a fixation element having a standard wire portion and a biodegradable, bioabsorbable or bioresorbable portion. FIG. 3A illustrates a temporary fixation element that may have a number of different configurations of both standard materials and degradable materials. The fixation element comprises a non-degradable (e.g., standard wire) portion 302, a degradable plug 304, and a hook 306. The hook can be a resorbable hook or a non-degradable (e.g., standard wire) hook. FIG. 3B illustrates a temporary fixation element 310 crimped to a support member 312 of a medical device. In the various embodiments that follow, there is a mating of a standard biocompatible material (e.g., portion 314) and a degradable material (e.g., portion 316) or resorbable material (e.g., portion 318). The junction between these two materials may be adjusted relative to the end of the crimp 320 (see FIG. 3B). In various embodiments, the junction between standard and degradable material may be under the crimp, in line with the edge of the end of the crimp or beyond the end of the crimp.

In one configuration of the illustrative embodiment of FIG. 3A, there is a standard anchor body and anchor tip each made of a suitable biocompatible metal. Between these two standard elements is a biodegradable, bioabsorbable or bioresorbable portion (e.g., plug 304). After implanting the medical device, the degradable portion is designed to degrade after the time during which tissue incorporation has occurred. At this point, the medical device frame is being held in place using predominantly the tissue incorporation force. The time of degradation of the portion between the two standard elements is selected to occur after this point. As a result, there is a portion of the leg of the fixation element within the crimp and a portion of the tip incorporated within the lumen wall.

In addition or alternatively, FIG. 3B also provides temporary fixation elements having different configurations. In one configuration, there is only a standard anchor body made of a suitable biocompatible metal with one or more anchor sections or tips that may degrade after incorporation. Beyond the standard fixation element portion 314 are one or more biodegradable, bioabsorbable or bioresorbable portions 316, 318. After implanting the medical device, at least one of the degradable portions is designed to degrade after the time during which tissue incorporation has occurred. At this point, the medical device frame is being held in place using predominantly the tissue incorporation force. The time of degradation of the portion between the standard element and one or more degradable portions is selected to occur after this point. As a result, there is a portion 314 of the leg of the fixation element within the crimp 320 and a portion of the tip incorporated within the lumen wall and optionally, another portion (e.g., portion 316) that degraded initially to separate from the crimp. In one embodiment, a degradable portion could be distal to the standard fixation portion within the crimp. This portion could be designed to degrade so as to separate the tip portion within the tissue from the rest of the fixation element. In one embodiment, the degradable portion of the fixation element within the tissue wall is resorbable into the lumen wall while the other potion between the lumen wall and the crimp is biodegradable and absorbed by the body.

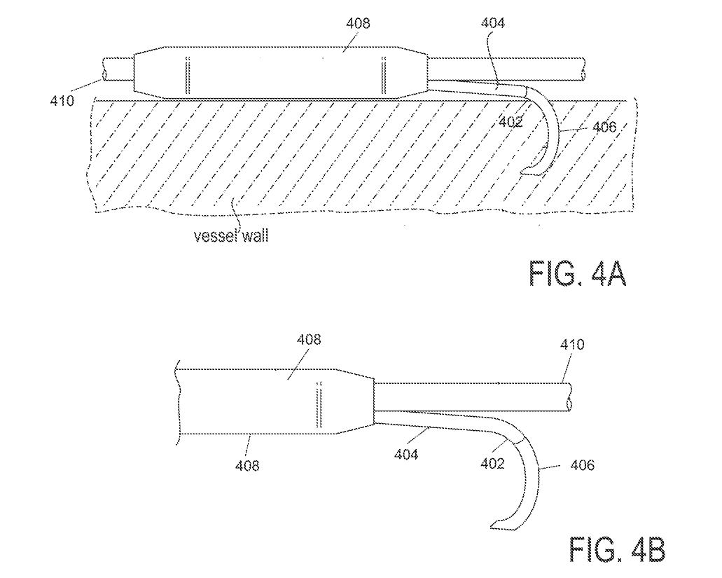

FIG. 4A illustrates a fixation element attached to a support frame 410 with a biodegradable, bioabsorbable or bioresorbable portion 402 engaged with the side wall of lumen. The portion 406 in the side wall of the lumen can be configured to resorb into the tissue after a given amount of time. The degradable portion 404 not positioned in the side wall can be configured to degrade after a period of time. FIG. 4B illustrates an enlarged view of the tissue engagement element of FIG. 4A with a biodegradable, bioabsorbable or bioresorbable tip portion comprising portions 404, 406, extending beyond the crimp. In one configuration, there is only a standard anchor body made of a suitable biocompatible metal with one tip that may degrade after incorporation. In the illustrated embodiments of FIGS. 4A and 4B, attached to a standard fixation element (e.g., positioned within crimp 408) is a biodegradable, bioabsorbable or bioresorbable section that terminates in a tip. After implanting the medical device, the degradable portion is designed to degrade after the time during which tissue incorporation has occurred.

In one aspect, tissue incorporation provides the mode to “fix” a filter or a portion of a filter in a desired location in a lumen. In one embodiment, the strength degradation curve of the biodegradable, bioabsorbable or bioresorbable portion engaged with the side wall of lumen is selected to occur at or after the time frame for tissue growth or tissue incorporation or encapsulation of the filter. In one aspect, the strength degradation curve of the biodegradable, bioabsorbable or bioresorbable portion engaged with the side wall of lumen is selected to occur three weeks after implantation or occur four weeks after implantation.

At this point (i.e., after strength degradation of the biodegradable, bioabsorbable or bioresorbable portion engaged with the side wall of lumen), the medical device frame is being held in place using predominantly the tissue incorporation force. The time of degradation of the portion between the standard element and degradable portion is selected to occur after this point. As a result, there is a portion of the leg of the fixation element within the crimp and a portion of the tip incorporated within the lumen wall. In one embodiment, a degradable portion could be distal to the standard fixation portion within the crimp. This portion could be designed to degrade so as to separate the tip portion within the tissue from the rest of the fixation element. In one embodiment, the degradable portion of the fixation element within the tissue wall is resorbable into the lumen wall while the other portion (if any) between the lumen wall and the crimp is biodegradable and absorbed by the body.

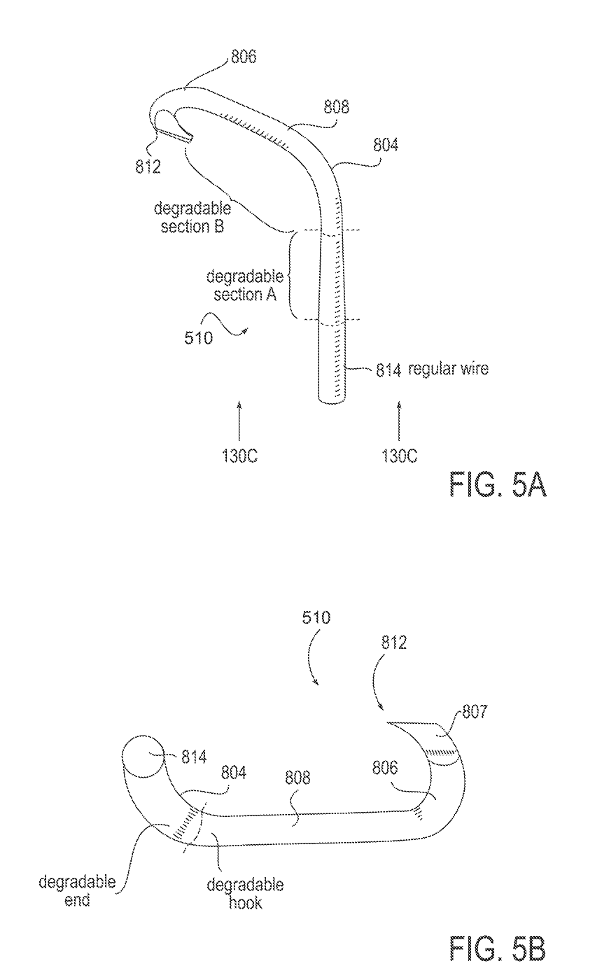

FIG. 5A is a perspective view of an extended anchor 510 shown in the first loop of the filter of FIG. 2 having a biodegradable, bioabsorbable or bioresorbable section A and B. The characteristics of this embodiment may be varied as described above with regard to FIGS. 3A, 3B, 4A and 4B. FIG. 5B is a side view of an alternative tip for the extended anchor of FIG. 5A. The embodiment of FIG. 5B may also be varied as described above. The anchors illustrated in FIGS. 5A and 5B may be used in a filter configuration similar to that of FIG. 2. In this alternative embodiment, there are temporary fixation elements on the portion of the filter having the material capture structure portion. The second loop of the filter (i.e., the loop without a material capture structure) would be configured to have permanent fixation elements. In other embodiments, there are temporary fixation elements on the portion of the filter without a material capture structure, and permanent fixation elements on the loop with the material capture structure. In other embodiments, both loops have permanent fixation elements. In some embodiments, both loops have temporary fixation elements. Other configurations of fixation element style and permanence may be provided to the filter embodiment of FIG. 2 and other structures described herein.

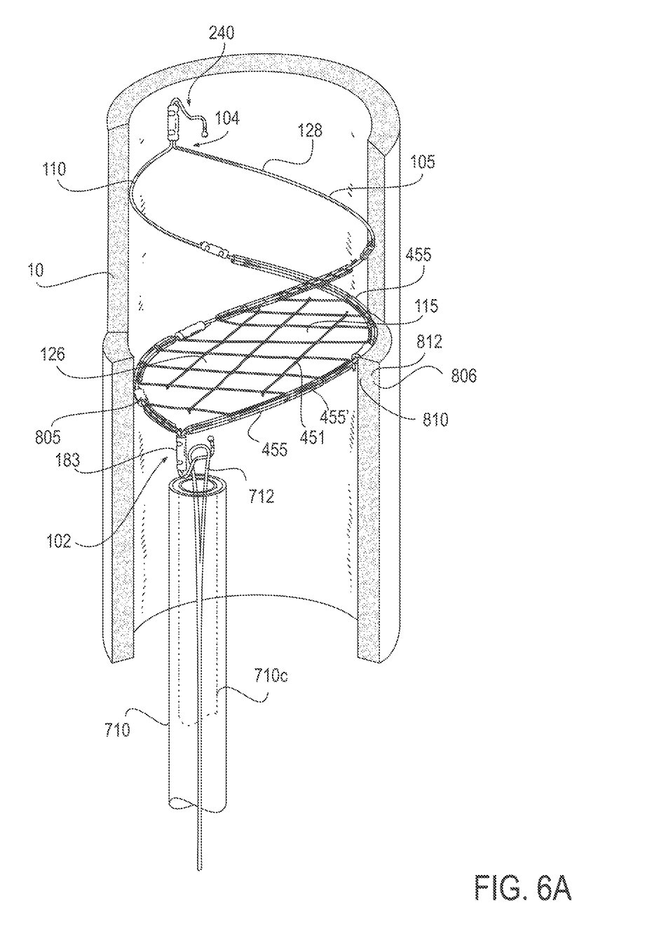

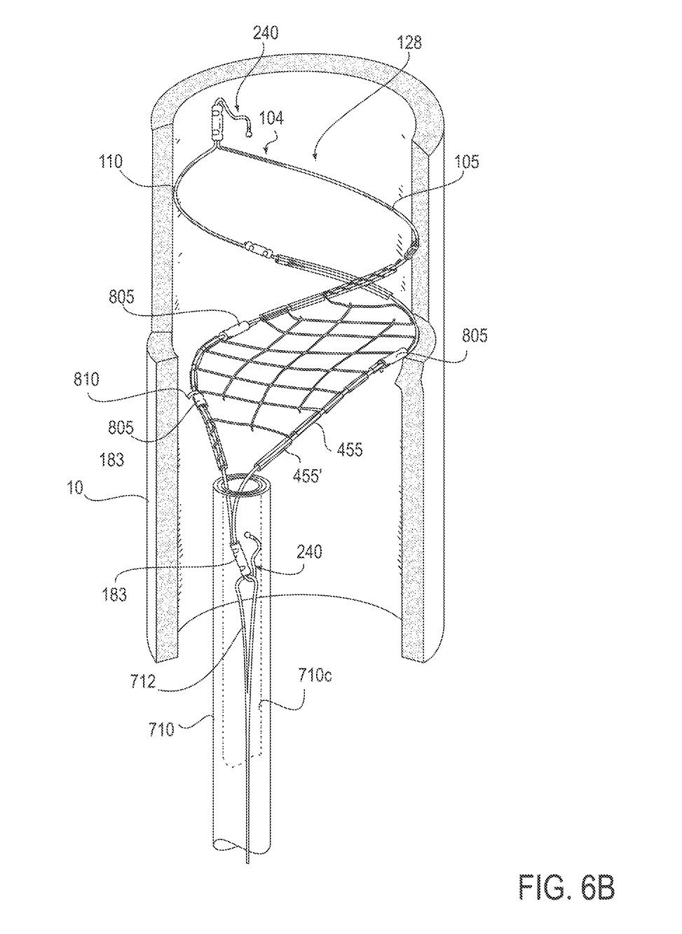

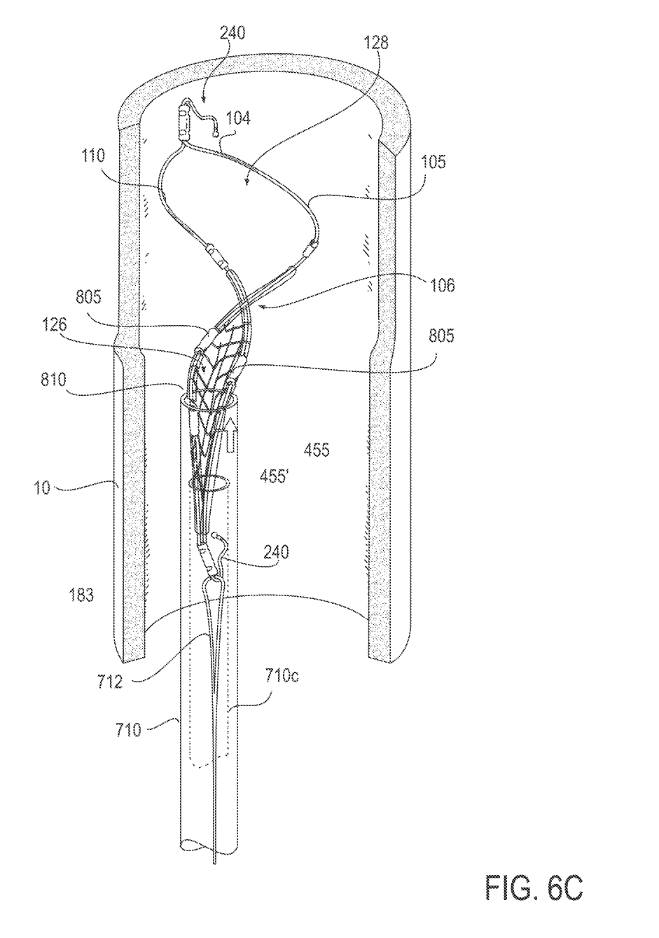

FIGS. 6A, 6B and 6C illustrate a section view of a vessel with the filter of FIG. 2 disposed therein. In this illustrative embodiment, the filter has passed the point where incorporation has occurred and the incorporated tissue is a contributing force to maintaining the filter in place. As before, the first step involves capture of the device retrieval feature (FIG. 6A). Next as in FIG. 6B, as the retrieval sheath is advanced over the support frame the permanent fixation elements of the first loop are pulled away from engagement with the walls. Here the removal force is not only the incorporated tissue but also the holding force of the fixation elements. It is important to note that by this stage of implantation, the temporary fixation elements in the second loop are no longer present having since degraded by this time. As a result, as the second stage of retrieval begins and the sheath moves to disengage the second loop, the force required is only that of the incorporated tissue. As a result, appropriate selection of the degradation profile of the temporary fixation elements required reduced effort to complete the retrieval operation of the mixed use filter of FIGS. 6A, 6B and 6C.

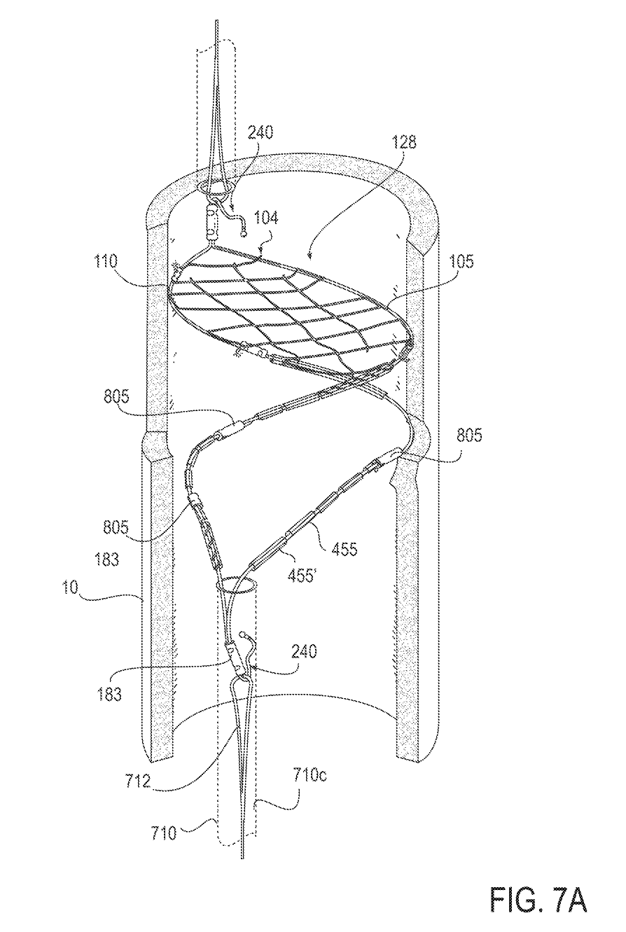

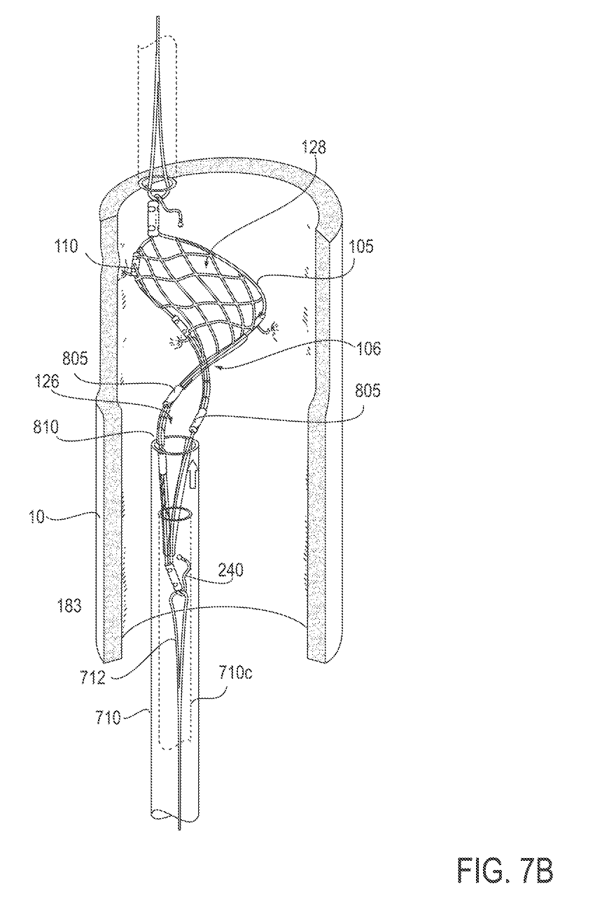

Similar to FIGS. 6A, 6B and 6C, FIGS. 7A and 7B illustrate two stages of the retrieval of either end of a filter having biodegradable, bioabsorbable or bioresorbable anchors depending upon degree of incorporation. Depending upon a number of factors, a user may determine that retrieval should proceed from the loop having the permanent anchors first. In this instance, recovery proceeds using the phantom snare and recovery sheath indicated in FIGS. 7A and 7B. In this case, the material capture structure will also be recovered first. In contrast, if recovery were to proceed from the loop having only temporary fixation elements, then recovery would precede using the solid snare and sheath shown in FIGS. 7A and 7B. In this instance, the lower force loop is recovered first so that more of the filter is within the recovery sheath when the higher pull forces are applied to remove the loop having the permanent fixation elements.

In still another embodiment, the retrieval force for a filter may be reduced through the use of a removable over layer on the support frame. As tissue incorporation occurs, the filter support frame is encapsulated against the tissue wall. In embodiments with the removable outer layer, the tissue is growing over and on the outer layer. The outer layer may be provided with one or more perforated sections that are adapted and configured to separate under the movement of the support frame to retrieval forces. In one aspect, the perforated or preferred separation region(s) are placed along an inner radius. Optionally or additionally, the perforated or break away sections are positioned such that when retrieval forces are applied to the support frame and the frame begins to collapse inwards (see FIG. 7B), the support frame movement against the outer layer opens the perforated portion. Since the outer layer has been incorporated into the lumen wall, as the perforated portion opens the support frame is released from the lumen using less force than the incorporation force. While desiring not to be bound by theory, it is believed to be more efficacious to employ lower recovery force retrieval that does not involve removal of or breaking incorporated tissue—filter layers. After filter recovery, the remaining incorporated outer layer remains in the lumen and continues to be incorporated into the lumen wall. Optionally, the outer layer is made from a biodegradable, bioabsorbable or bioresorbable material as described elsewhere herein.

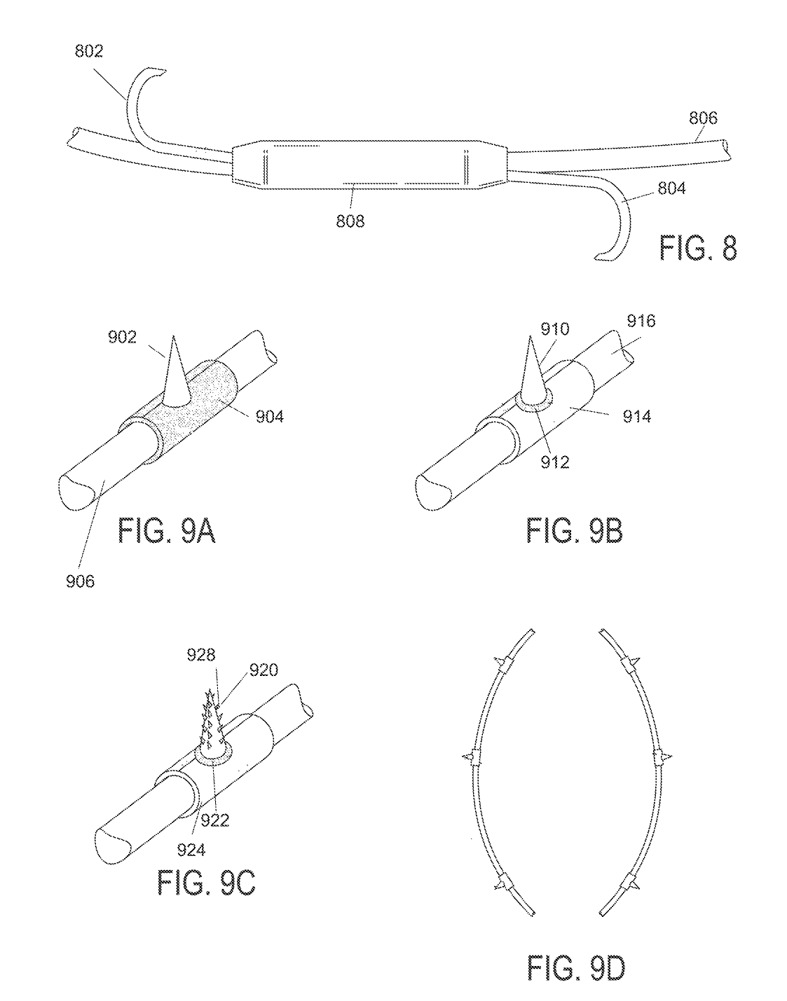

While many of the above embodiments had only one fixation element, the invention is not so limited. Embodiments of the temporary fixation elements of the present invention may take on any of the various configurations described herein. For example, FIG. 8 illustrates a double ended biodegradable, bioabsorbable or bioresorbable fixation element with different tip orientations attached to an elongate body. Tip 802 extends away from the support frame wire 806 in a first direction. Tip 804 extends away from the support frame wire in another direction. The fixation element can be attached to the support frame wire 806 using a crimp 808.

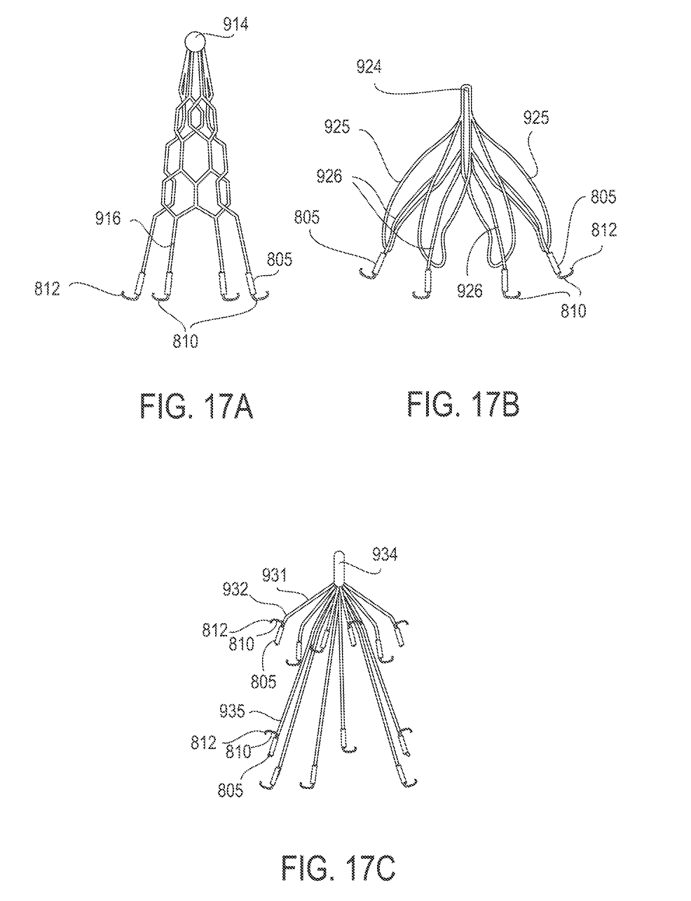

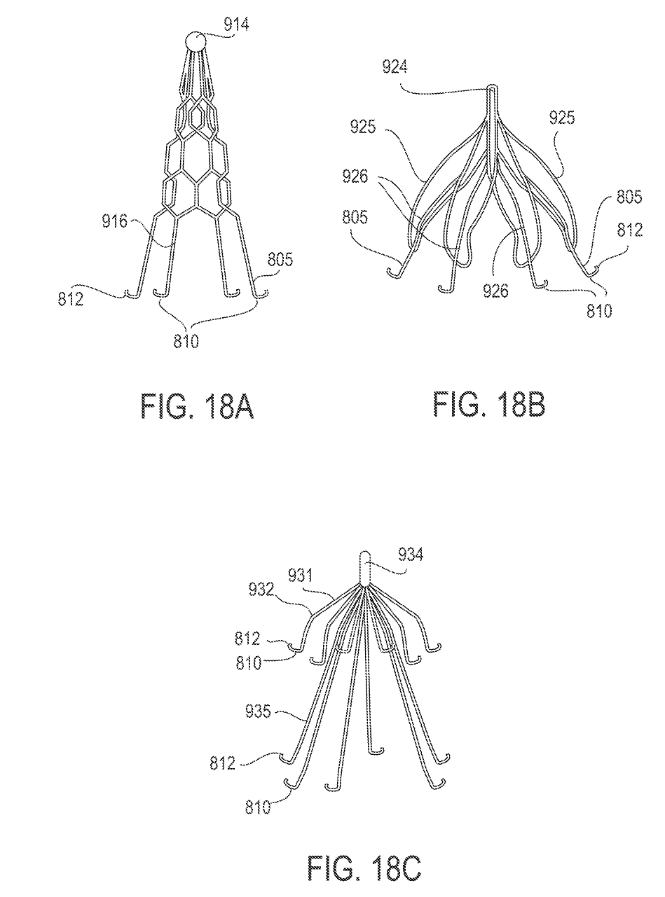

Still further variations of the temporary fixation elements are possible. For example, some fixation elements may be crimped to terminal ends of a structure. FIGS. 17A-17C illustrate crimped temporary fixation elements 810 near or proximate to a terminal portion of a support structure. Still further, some medical devices may have temporary fixation elements integrally formed with a portion of a structure. See for example, FIGS. 18A-18C where the terminal ends are biodegradable, bioabsorbable or bioresorbable fixation elements 810. In addition or alternatively, the temporary fixation elements location is selected based upon factors such as the rate and location of tissue incorporation, the dwell time of the medical device, the availability of suitable sites for biodegradable, bioabsorbable or bioresorbable actions to occur.

In still other configurations, the temporary fixation element may be attached to the medical device using a variety of different techniques. FIGS. 9A-9C illustrate a variety of tube and tip configurations for use as temporary fixation elements. FIG. 9B illustrates a tissue anchor 910 having a biodegradable, bioabsorbable or bioresorbable base 912 attached to a tube 914 (e.g., polymer tube) that is attached to a support member 916. The tissue anchor 910 can comprise non-degradable materials (e.g., polymer). In this embodiment, after the tissue incorporation period has passed, the degradation of the base portion will cause the tip 910 to separate from the tube 914. Thereafter, since the tissue incorporation period has passed, the tip 910 will remain safely incorporated in the tissue wall.

In contrast to the tube and tip of FIG. 9B, FIG. 9A illustrates a tissue anchor 902 on a biodegradable, bioabsorbable or bioresorbable tube 904 that is mounted on a support member 906. The tissue anchor 902 can comprise a non-degradable material (e.g., a polymer). In this embodiment, after the tissue incorporation period has passed, the degradation of the tube portion will cause the tip 902 to separate from the support member.

FIG. 9C illustrates a combination of the features of FIGS. 9A and 9B. In this embodiment, there is a biodegradable, bioabsorbable or bioresorbable tissue anchor 920 coupled to the tube 924 using a degradable base connection 922, like the base in FIG. 9B. The tissue anchor 920 can comprise thorns 928, as shown in FIG. 9C. Some or all of the thorns 928 can also comprise biodegradable, bioabsorbable, or bioresorbable materials. As with FIG. 9B, the time constant for degrading the degradable base connection is longer than the tissue incorporation time. Thereafter, the tissue anchor is separated from the tube. The separated tissue anchor will then proceed to degrade along the degradation time scale as it is absorbed or resorbed into the tissue.

FIG. 9D illustrates a plurality of the tissue anchors illustrated in any of the FIGS. 9A, 9B or 9C positioned along a pair of support structures.

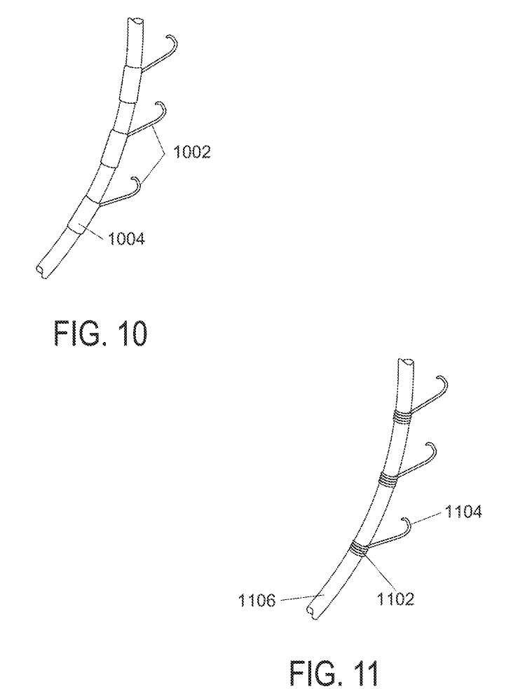

FIG. 10 is a variation of FIGS. 9A, 9B and 9C where the various types of degrading structures are used with the tube 1004. In addition, the shark tooth or thorn embodiments of FIGS. 9A-9C are replaced with hook shaped fixation elements 1002. Other shapes of fixation elements may also be combined with the tube illustrated in FIG. 10 and utilizing the various combinations of degrading properties described herein.

In contrast to the tube described in FIGS. 9A-9C and 10, FIG. 11 is a variation of a temporary fixation element support that replaces the tube described above with a plurality of wraps or coils 1102 about the support frame 1106. FIG. 11 illustrates yet another alternative use of a biodegradable, bioabsorbable or bioresorbable base structure to use in temporary fixation elements. Alternatively, the fixation element 1104 could be biodegradable with the wraps or coils 1102 being non-degradable.

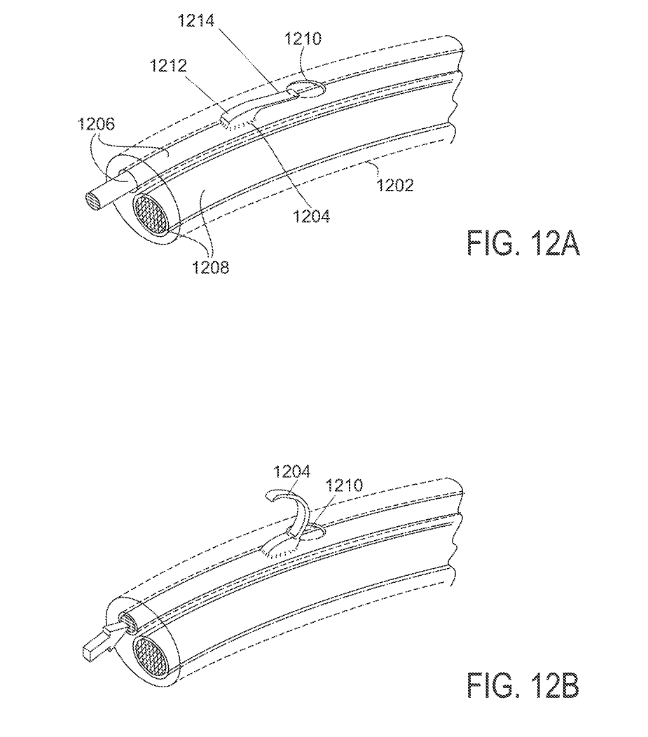

Temporary fixation elements may also be provided using retractable fixation elements. In one embodiment, a dual lumen cover is used for both the support frame in one lumen and for an anchor actuator in another lumen. FIGS. 12A and 12B illustrate section and perspective views respectively of a dual lumen sleeve or coating 1202 enclosing a support member 1208 and an anchor support wire 1206. FIG. 12A shows (in phantom) how a biased outward anchor element 1204 is maintained within the lumen of the covering. The anchor 1204 has a fixed end 1212 and a free, biased end 1214. When advanced, an anchor extends out of an anchor port 1210 in the sidewall of the covering as shown in FIG. 12B.

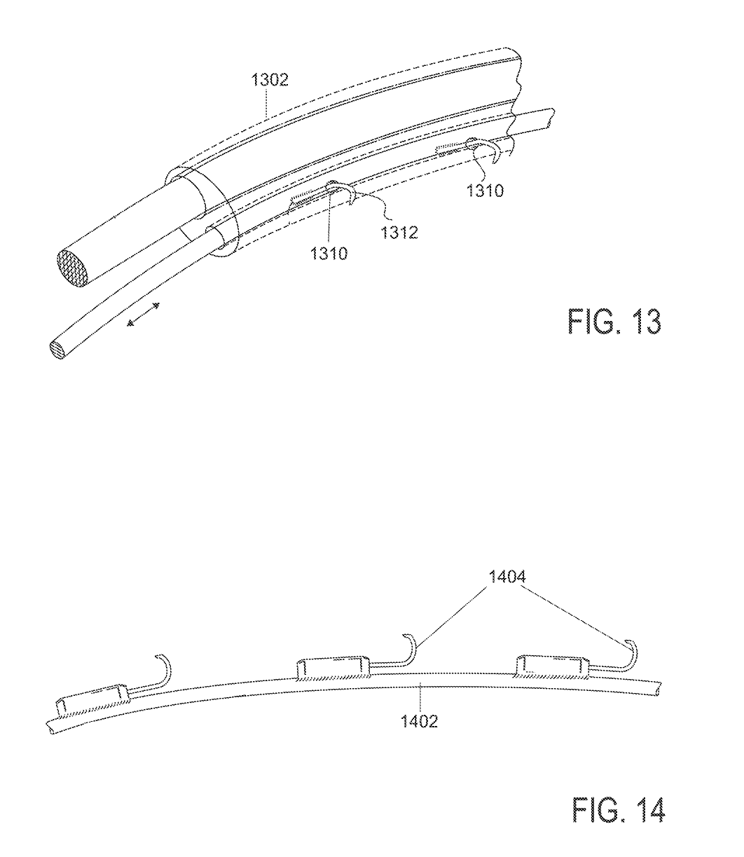

There may be more than one anchor port along the device. FIG. 13 is a dual lumen sleeve 1302 as in FIGS. 12A and 12B having multiple ports 1310 with anchors 1312 have a fixed and a free, biased end. FIG. 14 is a perspective view of an anchor wire with three anchors 1404 for use in a multiple anchor configuration. The number, size, orientation and spacing of the anchors along the medical device may be varied depending upon the desired anchor configuration.

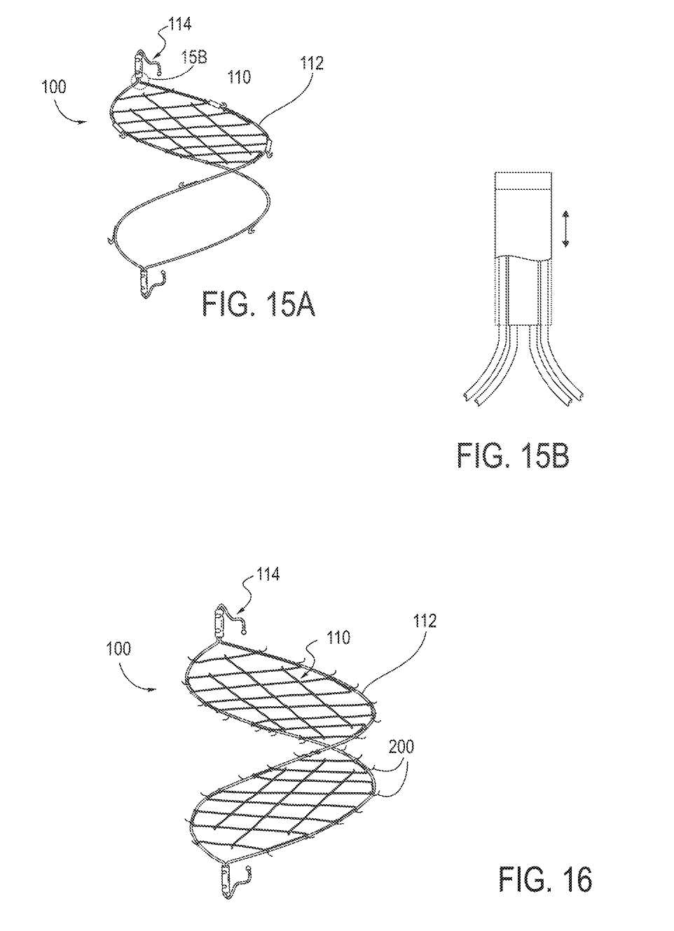

FIG. 15A is a perspective view of an endoluminal filter with three retractable anchors and three fixed anchors. FIG. 15B is an illustrative embodiment of a retraction and extension device on one end of the filter. Operation of the device causes the anchors to retract into or deploy from the anchor ports in the lumen.

FIG. 16 is a perspective view of a dual loop filter having multiple mixed retractable or biodegradable, bioabsorbable or bioresorbable fixation elements.

FIGS. 17A, 17B and 17C illustrate various filters having one or more biodegradable, bioabsorbable or bioresorbable anchors 810 as described in FIGS. 3A-4B.

FIGS. 18A, 18B, and 18C illustrate various filters having one or more biodegradable, bioabsorbable or bioresorbable anchors 810 integrally formed with the filter as described in FIGS. 5A and 5B.

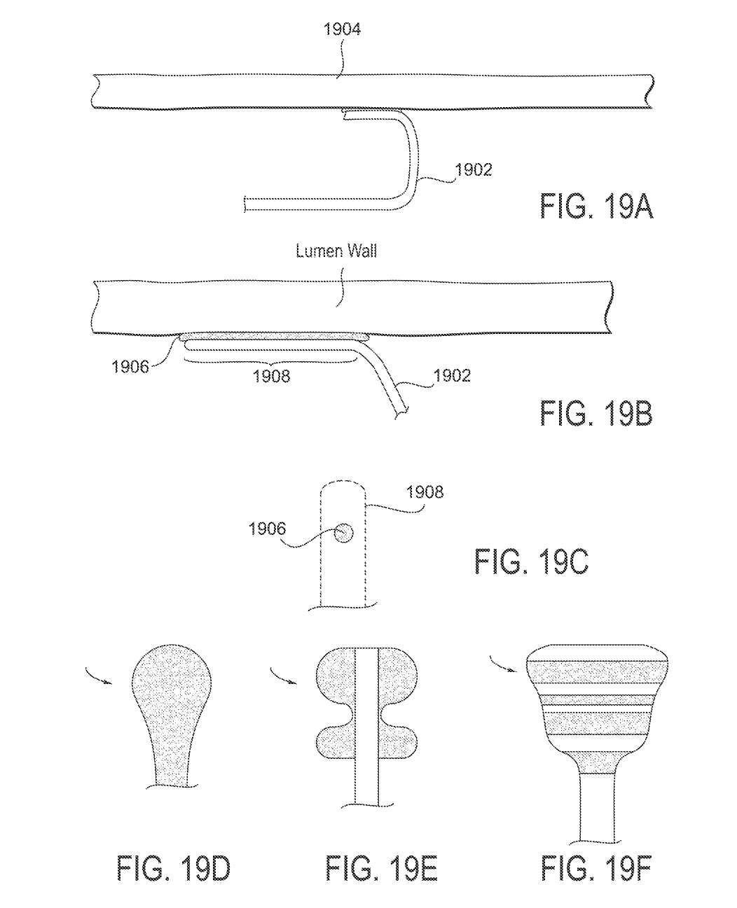

FIG. 19A is a side view of a non-penetrating anchoring member 1902 positioned against a lumen wall 1904. As seen in this view, the distal end of the anchoring member lies against the lumen wall. Optionally or additionally, the anchoring member may be configured as illustrated and described above. FIG. 19B is an enlarged view of FIG. 19A. FIG. 19B illustrates the adhesive layer 1906 that is on the distal end of the anchoring member 1902. The adhesive layer 1906 is positioned along the anchoring member at engagement section 1908 to further the engagement between the anchoring member and the wall portion. FIG. 19C is a top down view of the distal most end of the anchoring member of FIGS. 19A and 19B. In this embodiment, the distal most end is coated with an adhesive layer 1906 that will bond with the inner portion of the lumen wall. The adhesive layer may be any bonding agent or glue. In one embodiment, the adhesive layer is a medical grade biocompatible adhesive. In one aspect, the adhesive layer is coated with a non-sticky layer to prevent the adhesive layer from sticking to other surfaces in filter or delivery system. The non-sticky layer may dissolve when exposed to the lumen wall or fluids thereby exposing the adhesive layer. Optionally, the non-sticky layer may be removed prior to deployment of the anchoring member.

The generally rectangular perimeter of the anchoring member is also shown in the top down view of FIG. 19C. In addition, the adhesive layer completely covers the distal most end. Other configurations of both the perimeter and the distribution of the adhesive layer are illustrated in FIGS. 19D, 19E and 19F. FIG. 19D illustrates a bulb shaped distal end. Like the illustrative embodiment of FIG. 19C, the adhesive layer completely covers the distal most end that will be in contact with the lumen wall. FIG. 19E illustrates a top down view of another alternative configuration of the distal most end of the anchoring member. In this embodiment, the shape of the distal most end has a FIG. 8, double lobe or butterfly shaped perimeter. In this embodiment, in contrast to the previous embodiments, the adhesive layer is only along the edges of the lobes. The central portion of the anchoring member does not have an adhesive layer. FIG. 19F illustrates a top down view of another alternative configuration of the distal most end of the anchoring member. In this embodiment, the shape of the distal most end has a rounded trapezoid shaped perimeter. In this embodiment, in contrast to the previous embodiments, the adhesive layer is placed along the upper surface in a pattern. In the illustrated embodiment, the adhesive layer is made up of a pattern of adhesive layers of various widths. The pattern of adhesive layers may also be spaced apart by uniform or non-uniform spacing. In another embodiment, the adhesive pattern is made of regularly shaped and sized stripes of uniform spacing.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An endoluminal filter, comprising:
a first support member having a first end and a second end;
a second support member attached to the first end of the first support member or the second end of the first support member and forming a crossover with the first support member;
a material capture structure extending between the first and second support members, the crossover and the first end or the second end of the first support member;
at least one permanent tissue anchor on the first support member or the second support member used to support the material capture structure; and
at least one degradable tissue anchor on the first support member or the second support member adjacent to the portion of the filter without the material capture structure.

2. An endoluminal filter according to claim 1 wherein the second support member is attached to the first end of the first support member and the second end of the first support member.

3. An endoluminal filter according to claim 1 wherein the first support member forms a tissue anchor and the second support member forms a retrieval feature.

4. An endoluminal filter according to claim 1 further comprising:
a retrieval feature on the first end and a retrieval feature on the second end.

5. An endoluminal filter according to claim 1 further comprising a combined tissue anchor and retrieval feature joined to the first end or the second end of the first support member.

6. An endoluminal filter according to claim 1 further comprising:
an attachment element that joins the first support member to the second support member.

7. An endoluminal filter according to claim 6 the attachment element further comprising a tissue anchor.

8. An endoluminal filter according to claim 1 wherein the at least one permanent or degradable tissue anchor is formed on or attached to a surface of the first support member or the second support member.

9. The endoluminal filter according to claim 8 wherein the tissue anchor is attached to the first support member or the second support member by a crimp.

10. The endoluminal filter according to claim 8 wherein the tissue anchor is attached to the first support member or the second support member by a suitable joining technique.

11. The endoluminal filter according to claim 10 wherein the portion of the tissue anchor beyond the portion attached to the first support member or the second support member is degradable.

12. An endoluminal filter according to claim 1 wherein the at least one permanent or degradable tissue anchor on the first support member or the second support member is positioned between the crossover and the first end or the second end.

13. An endoluminal filter according to claim 1 wherein the at least one permanent or degradable tissue anchor comprises more than one tissue anchor at a location on the first or second support member.

14. An endoluminal filter according to claim 1 wherein the at least one permanent or degradable tissue anchor is formed from or attached to a tube covering at least a portion of the first support member or the second support member.

15. An endoluminal filter according to claim 1 wherein the at least one permanent or degradable tissue anchor is a tube having a tissue engagement surface.

16. The endoluminal filter of claim 15 wherein the tube is permanent and the tissue engagement surface is degradable.

17. An endoluminal filter according to claim 15 wherein the tissue engagement surface comprises a raised form.

18. The endoluminal filter of claim 17 wherein the raised form is a triangular shape, an apex, a conical shape, or a form having jagged edges.

19. The endoluminal filter of claim 18 wherein the tube is permanent and the raised form is degradable.

20. An endoluminal filter according to claim 17 wherein the raised form comprises a spiral form.

21. An endoluminal filter according to claim 1 wherein the tissue anchor comprises a coil wrapped around the first or the second support member having at least one end adapted to pierce tissue.

22. A filter, comprising:
a first support member having a first end and a second end;
a second support member having a first end and a second end;
a filter structure suspended within a first loop formed by the first support member and the second support member between a point where the first end of the first support member joins the first end of the second support member and a point where the first support member crosses without being joined to the second support member;
a permanent tissue anchor borne by the first loop on the first support member or the second support member; and
a degradable tissue anchor within a second loop formed by the first support member and the second support member between a point where the second end of the first support member joins the second end of the second support member and a point where the first support member crosses without being joined to the second support member.

23. An endoluminal filter having retractable fixation elements, comprising:
a first multiple lumen support member having a first end and a second end;
a second multiple lumen support member attached to the first end of the first support member or the second end of the first support member and forming a crossover with the first support member;
a material capture structure extending between the first and second support members, the crossover and the first end or the second end of the first support member;
a first fixation element support disposed within a lumen of the first multiple lumen support member;
a second fixation element support disposed within a lumen of the second multiple lumen support member;
at least one tissue anchor supported by and moveable with the first fixation element support disposed within a lumen of the first multiple lumen support;
at least one tissue anchor supported by and moveable with the second fixation element support disposed within a lumen of the second multiple lumen support;
At least one opening in the sidewall of the lumen of the first multiple lumen support member adapted and configured to permit the deployment and retrieval of the at least one tissue anchor borne by the first fixation element support disposed within a lumen; and
At least one opening in the sidewall of the lumen of the second multiple lumen support member adapted and configured to permit the deployment and retrieval of the at least one tissue anchor borne by the second fixation element support disposed within a lumen.

24. An endoluminal filter having retractable fixation elements, comprising:
a first support member having a first end and a second end;
a second support member attached to the first end of the first support member or the second end of the first support member and forming a crossover with the first support member;
a material capture structure extending between the first and second support members, the crossover and the first end or the second end of the first support member;
a first fixation element support disposed within a lumen of the first multiple lumen support member;
a second fixation element support disposed within a lumen of the second multiple lumen support member;
at least tissue anchor supported by and moveable with the first fixation element support disposed within a lumen of the first multiple lumen support;
at least tissue anchor supported by and moveable with the second fixation element support disposed within a lumen of the second multiple lumen support;
a first covering over the first support member and the first fixation element support having at least one opening in a sidewall of the first covering adapted and configured to permit the deployment and retrieval of the at least one tissue anchor borne by the first fixation element support; and
a second covering over the second support member and the second fixation element support having at least one opening in a sidewall of the second covering adapted and configured to permit the deployment and retrieval of the at least one tissue anchor borne by the second fixation element support.

25. An endoluminal filter having retractable fixation elements according to claim 24 further comprising: a lumen within the first covering adapted and configured to house the first support member and the first fixation element support and a lumen within the second covering adapted and configured to house the second support member and the second fixation element support.
